(19) United States
(12) Patent Application Publication
(10) Pub. No.: US 2018/0310846 A1
(43) Pub. Date: Nov. 1, 2018

(54) A REFLECTION-TYPE PULSE DETECTION DEVICE AGAINST MOTION INTERFERENCE

(71) Applicant: SHANGHAI MEGAHEALTH TECHNOLOGIES CO., LTD, Shanghai (CN)

(72) Inventor: Wei LIN, Shanghai (CN)

(21) Appl. No.: 15/770,331

(22) PCT Filed: Nov. 26, 2015
(86) PCT No.: PCT/CN2015/095650

§ 371 Date: Apr. 23, 2018

(30) Foreign Application Priority Data
Sep. 22, 2015 (CN) 201510605384.4

(51) Int. Cl. A61B 5/024; A61B 5/00; G16H 50/20

(52) U.S. Cl. CPC A61B 5/02427; G16H 50/20; A61B 5/7207; A61B 5/002

(57) ABSTRACT

Disclosed is a reflection-type pulse rate detection device against motion interference, which relates to the technical field of the pulse rate detection device. The device includes a reflection type pulse rate detection module (1), a circuit driving module (2), a signal conditioning module (3), an ultra-low power wireless transceiver module (4), a microprocessor control module (5), a display module (6) and a remote terminal (7). The reflection-type pulse rate detection module (1), which is connected with the circuit driving module (2), consists of a first LED light tube (101), a second LED light tube (103) and a photoelectric receiving tube (102). The signal conditioning module (3) consists of a detection circuit (301), a filter circuit (302) and a subtraction circuit (303). The detection circuit (301), the filter circuit (302) and the subtraction circuit (303) are connected to the microprocessor control module (5) in turn, which is respectively connected with the LED circuit driving module (2), the ultra-low power wireless transceiver module (4) and the display module (6). The ultra-low power wireless transceiver module (4) is connected with the remote wireless terminal (7). The present invention with the ability of motion interference reduction is low consumption and can interact with the remote terminal. Extracting the pulse rate signal with higher SNR and suppressing common motion interference, it has strong anti-saturation ability.

### TECHNICAL FIELD

The invention relates to the technical field of the pulse rate detecting device, which is specifically related to a reflection-type type pulse detection device against motion interference.

### BACKGROUND ART

With the improvement of people's economic conditions, the health care needs and treatment measures have developed obviously. The numbers of the patients suffering from cardiovascular and cerebrovascular diseases which have become one of the main causes of human death are still rising year by year. Therefore, it's necessary to understand, prevent and detect the early signs of these diseases.

A pulse rate detecting device is an electronic instrument used for detecting one's pulse rate or heart rate, which is an important part of the ECG, so it plays an important role in modern medicine. To a large extent, it can reflect the blood flow characteristics of many physiological pathology in human cardiovascular system, so it has very high medical value and application prospect. Currently on the market, there are a wide variety of instruments to detect the pulse rate, not many of that can detect the pulse rate precisely. It's mainly because that the biological signal of human body is weak signal in strong noise background. Moreover, pulse wave signal is non-electric physiological signal of low frequency, which must be magnified and filtered to meet the signal acquisition requirements. Normal device cannot reduce the motion interference and extract the pulse rate signal with higher SNR, and the ability of motion interference reduction and anti-saturation is limited.

In order to solve the problems mentioned above, it's necessary to design a pulse rate detection device with motion interference reduction.

### SUMMARY OF INVENTION

To overcome the shortcomings of the existing devices, this invention, which is simple structure, reasonable design, easy to use, low power consumption, aims at providing a reflection-type pulse rate detection device against motion interference, and it also has the ability to interact with the remote terminal and the ability of motion interference reduction and anti-saturation.

In order to realize the purposes mentioned above, the invention applies the following technical proposal: a reflection-type pulse rate detection device against motion interference which includes a reflection-type pulse detection module, and a LED circuit driving module, a signal conditioning module, a ultra-low power wireless transceiver module, a microprocessor control module, a display module and a remote terminal corresponding to the reflection-type pulse detection module. The reflection-type pulse rate detection module is connected with the circuit driving module, and consists of the first LED light tube, the second LED light tube and the photoelectric receiving tube. The signal conditioning module consists of a detection circuit, a filter circuit, and a subtraction circuit. The detection circuit, the filter circuit and the subtraction circuit are connected to the microprocessor control module in turn. The pulse wave analog signal is preprocessed by the detection circuit, the filter circuit and the subtraction circuit and enters into the microprocessor control module. The pulse wave signal with high SNR can be acquired at the analog end and the complexity of processing the digital signal can be reduced. And the microprocessor is respectively connected to the circuit driving module, the ultra-low power wireless transceiver module, and the display module. And the ultra-low power wireless transceiver module is connected to the remote terminal over the wireless link.

As a preferred embodiment, the signal conditioning module uses a dual-path detection circuit, one of which is a detecting circuit for pulse rate signal with the superimposition of motion noise, while the other path is a detecting circuit for the motion noise; processed with the appropriate algorithm, the motion interference is cancelled; the pulse wave signal with a higher SNR is extracted.

As a preferred embodiment, the combination manner of the first LED luminous tube and the second LED luminous tube is emitting monochromatic or double color light with first wavelength and second wavelength; for example, the first LED luminous tube and the second LED luminous tube are both Red/Green double color LED. The first wavelength is used for detecting pulse wave analog signal, and the second wavelength is mainly used for following the motion interference signal.

The invention with the ability of motion interference reduction is low consumption and can interact with the remote terminal. Extracting the pulse rate signal with higher SNR and suppressing common mode interference, it has strong anti-saturation ability and has good application prospect.

### DESCRIPTION OF DRAWINGS

Next, the invention will be illustrated in detail with the drawing and the concrete implementation method.

FIG. 1 shows the structure of the invention.

### DETAILED EMBODIMENT

In order to make the technical means, creation characteristic, purpose and effect of the invention easy to understand, the invention will be further illustrated with the following concrete implementation method.

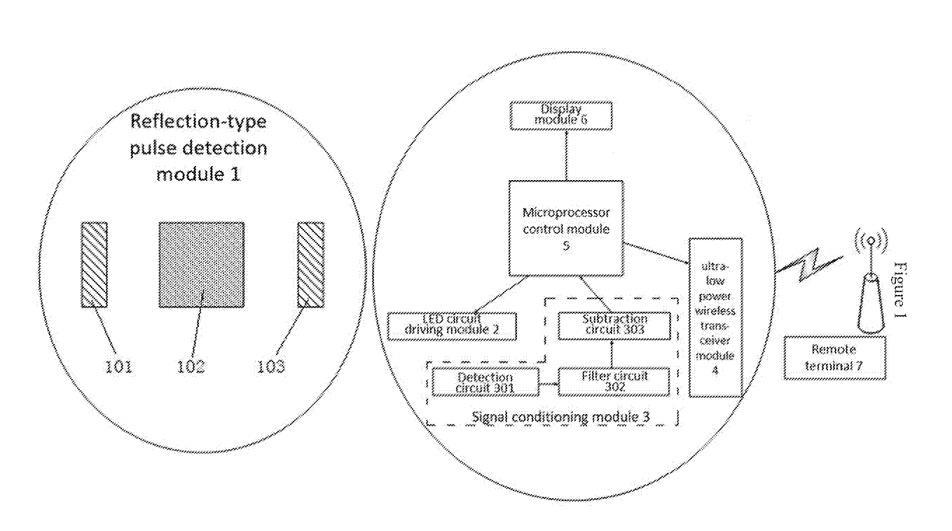

Referring to FIG. 1, the concrete implementation method adopts the following technical proposal: A reflection-type pulse rate detection device against motion interference which includes the reflection-type pulse detection module (1), circuit driving module (2), signal conditioning module (3), ultra-low power wireless transceiver module (4), microprocessor control module (5), display module (6) and remote terminal (7) corresponding to the reflection-type pulse rate detection device against motion interference. The reflection-type pulse rate detection module (1) is connected to the circuit driving module (2) and consists of the first LED light tube (101), the second LED light tube (103) and the photoelectric receiving tube (102). The signal conditioning module (3) consists of detection circuit (301), filter circuit (302), and subtraction circuit (303). Detection circuit (301), filter circuit (302) and subtraction circuit (303) are connected to the microprocessor control module (5) in turn. And the microprocessor control module (5) is connected to the LED circuit driving module (2), ultra-low power wireless transceiver module (4), and display module (6). And the ultra-low power wireless transceiver module (4) is connected to the remote terminal (7) over the wireless link.

It is worth noting that the first LED luminous tube and the second LED luminous tube emit monochromatic or double color light. The wavelengths are λ₁ and λ₂ respectively. For example we use a Red/Green double color LED, of that λ₁ is mostly used for detecting pulse wave analog signal, and λ₂ is mainly used for following the motion interference.

According to the detailed embodiments, the first LED luminous tube (101) and the second LED luminous tube (103) are controlled by the LED circuit driving module (2) to adjust the light intensity. λ₁ is mainly used for detecting pulse wave analog signal, and λ₂ is mainly used for following the motion interference.

According to the detailed embodiments, the signal conditioning module (3) uses a dual-path detection circuit, one path of which is a detecting circuit for the pulse rate signal with the superimposition of motion interference, while the other path of which is a detecting circuit for the motion interference. The algorithm including the software part and the hardware part is used to reduce the motion interference and extract the pulse wave signal with higher SNR. The pulse wave analog signal is preprocessed by detection circuit (301), filter circuit (302), and subtraction circuit (303) and then enters into the microprocessor control module (5). The common mode noise is suppressed. The pulse rate signal can be displayed locally on the display module (6), and through the ultra-low power wireless transceiver module (4), it can also be transferred to the remote terminal (7) for display. The device has an ability to interact with the remote terminal (7).

The detailed embodiment is applied to long time dynamic real-time acquisition of human pulse rate. The invention with the ability of interacting with the remote terminal, motion interference reduction and anti-saturation is low power consumption, simple structure and reasonable design. So it has great medicine value and broad market application prospect.

The basic principles, main features and the advantages of the invention are illustrated and described above. The embodiments set forth above are considered to be illustrative and not limiting. Various changes to the above-described embodiments may be made without departing from the spirit and scope of the invention. Various presently unforeseen or unanticipated alternatives, modifications, variations, or improvements therein may become apparent and subsequently made by those skilled in the art which are also intended to be encompassed by the following claims. The teachings of any printed publications including patents and patent applications are each separately hereby incorporated by reference in their entirety.

1. A reflection-type pulse rate detection device against motion interference is characterized, which includes a reflection-type pulse detection module (1), a circuit driving module (2), a signal conditioning module (3), an ultra-low power wireless transceiver module (4), a microprocessor control module (5), a display module (6) and a remote terminal (7); the reflection type pulse rate detection module (1) consists of a first LED light tube (101), a second LED light tube (103) and a photoelectric receiving tube (102); the circuit driving module (2), the signal conditioning module (3), the ultra-low power wireless transceiver module (4) and the display module (6) are all connected to the microprocessor control module (5); and the ultra-low power wireless transceiver module is connected to the remote terminal (7) over the wireless link.

2. The reflection-type pulse rate detection device against motion interference as described in claim 1, wherein, the signal conditioning module (3) consists of a detection circuit (301), a filter circuit (302) and a subtraction circuit (303), the detection circuit (301), the filter circuit (302) and the subtraction circuit (303) are connected to the microprocessor control module (5) in turn.

3. The reflection-type pulse rate detection device against motion interference as described in claim 1 wherein, the signal conditioning module (3) uses a dual-path detection circuit, one of which is a detecting circuit for pulse rate signal with the superimposition of motion noise, while the other path is a detecting circuit for the motion noise; processed with the appropriate algorithm, the ability of motion interference reduction is improved; the front-end analog circuit can acquire the pulse wave signal with a higher SNR.

4. The reflection-type pulse rate detection device against motion interference as described in claim 1 wherein, the combination manner of the first LED luminous tube (101) and the second LED luminous tube (103) is emitting monochromatic or double color light with different wavelength; a typical application is the first LED luminous tube (101) and the second LED luminous tube (103) are both Red/Green double color LED.

5. The reflection-type pulse rate detection device against motion interference as described in claim 1 wherein, the first LED luminous tube (101) and the second LED luminous tube (103), which emit two types of lights with different wavelengths, a first wavelength and a second wavelength, are controlled by the circuit driving module (2) to adjust the light intensity; the first wavelength is used for detecting pulse wave analog signal, and the second wavelength is mainly used for following the motion interference signal; the pulse wave analog signal is preprocessed by the detection circuit (301), the filter circuit (302) and the subtraction circuit (303), and enters into the microprocessor control module (5); the common mode noise is suppressed; the pulse rate signal can be displayed locally on the display module (6), or can also be transferred to the remote terminal (7) for display through the ultra-low power wireless transceiver module (4).
